(19) United States
(12) Patent Application Publication
(10) Pub. No.: US 2007/0274928 A1
(43) Pub. Date: Nov. 29, 2007

(54) Novel Cyclic Oxygenated Compounds Having Cooling, Fragrance, and Flavor Properties, and Uses Thereof

(76) Inventor: Sergey Selifonov, Plymouth, MN (US)

Correspondence Address: FISH & RICHARDSON P.C., PO BOX 1022, MINNEAPOLIS, MN, 55440-1022, US

(21) Appl. No.: 10/579,316

(22) PCT Filed: Nov. 17, 2004
(86) PCT No.: PCT/US04/38373

§ 371 Date: Feb. 15, 2007

Related U.S. Application Data
(60) Provisional application No. 60/520,380, filed on Nov. 17, 2003.
(60) Provisional application No. 60/547,572, filed on Feb. 26, 2004.

(51) Int. Cl. A61K 8/30; A23G 3/00; A23G 4/00; A23L 2/00; A61P 43/00; A61Q 13/00; A61Q 11/00; A24B 13/00; A61K 31/12; A61K 31/34

(52) U.S. Cl. 424/49; 131/352; 426/531; 426/590; 426/660; 512/11; 512/8; 514/470; 514/690

(57) ABSTRACT

Compositions that include compounds capable of producing a cooling sensory effect such as certain hydroxy-ketones and hydroxy-aldehyde compounds, their cyclic semi-ketals and semiacetals, and other derivatives thereof. The compounds impart a refreshing and cooling sensation of long duration and high potency, and thus are useful in a variety of formulations, including consumer products such as mouth formulations, food and beverage products, tobacco and smoking articles, fragrances, toiletries, ointments, and the like.

This application claims priority to Selifonov, U.S. Provisional Patent Application No. 60/520,380 entitled “Cyclopentane compounds and their use,” filed Nov. 17, 2003, and to Selifonov, U.S. Provisional Patent Application No. 60/547,572 entitled “Compounds posessing cooling sensory effect and their use”, filed Feb. 26, 2004, both of which are incorporated by reference in their entirety.

### BACKGROUND

Many compounds causing a cooling sensory effect akin to the well-known effect of menthol are known in the art. However, the duration of the cooling effect is often short-lived. In addition, such compounds often have relatively low potency. Thus, it is necessary to include relatively high amounts of such compounds in formulated products. In addition, many of these compounds are expensive to manufacture on an industrial scale. Therefore, novel compounds with high potency and long lasting cooling effect are highly sought by the flavor and fragrance industries for applications in a variety of consumer products.

### SUMMARY

Novel compounds capable of producing a cooling sensory effect such as certain hydroxy-ketones and hydroxy-aldehyde compounds, their cyclic semi-ketals and semiacetals, and other derivatives thereof capable of forming such compounds upon exposure to moisture, heat, light, solvents, acids, or bases are described. The compounds are remarkably effective in imparting a refreshing and cooling sensation of long duration and high potency, and thus are useful in a variety of formulations, including consumer products such as mouth formulations, food and beverage products, tobacco and smoking articles, fragrances, toiletries, ointments, and the like.

Also described are novel odoriferous compounds useful in fragrance and flavor applications that are synthesized from two oxygenated monoterpenes (2-oxo-4,5,5-trimethyl-cyclopent-3-eneneacetic and 3-oxo4,5,5-trimethyl-cyclopentaneacetic acids) using various carbon-carbon bond forming synthetic methods.

### DETAILED DESCRIPTION

Cyclic compounds are disclosed that have a very powerful menthol-like cooling effect when applied orally, inhaled, or applied topically to the skin, or on an internal epithelium of a human or animal body. In particular, it has been found that certain novel hydroxylated carbonyl (aldehyde or ketone) compounds, and their derivatives, have an exceptionally long lasting and extremely powerful cooling effect that makes them useful in a number of applications, including as flavorings in chewing gum, confectionary, beverages, toothpaste, mouth wash, smoking and chewing tobacco articles, perfume, cosmetics, skin care and skin cleaning products, air fresheners, cleaning towels, clothing, topical medicinal preparations, formulations for the relief of hemorrhoid symptoms and irritable bowel syndrome, and other applications where a cooling sensory effect is desired.

The hydroxylated carbonyl compounds are represented by a group of compounds capable of forming either a cyclic semi-ketal or a cyclic semi-acetal ring having 5, 6, or 7 (preferably, 5 or 6) atoms forming the ring, or a cyclic vinyl ether having the same number of atoms forming the ring. In particular, one set of the compounds is represented by formulae (1) and (2):

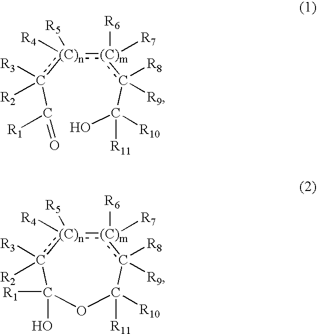

wherein R₁ through R₁₁ are each independently selected from H, linear or branched alkyl, alkenyl, or alkylidene groups having from 1 to 6 carbon atoms, or cycloalkyl or cycloalkenyl groups having from 3 to 10 carbon atoms. At least one of the R₁ through R₁₁ groups may further include a group selected from hydroxyl, amino, carboxyl, and carboxamide groups. In addition, any two R groups may be connected to each other by one or more carbon atoms other than those shown in the formulae, or by a heteroatom selected from oxygen, nitrogen, or sulfur atoms. Any one of the dashed bonds may be single or double, but no more than one dashed bond is double, and no two adjacent bonds in the structures are double at the same time. Furthermore, n and m are each independently 0 or 1, and the total number of carbon atoms in the compound is less than 20.

Depending on the particular compound, these compounds may exist preferentially as mixtures of open (formula 1) and cyclic (formula 2) forms, or as substantially pure compounds. Factors such as solvent conditions, pH, temperature, and the presence of other chemical compounds may influence the relative amounts of open (1) and cyclic (2) forms. For purposes of practicing the present invention, both open and cyclic forms, and their mixtures, are useful for imparting the desired cooling sensation. In general, compounds in the cyclic form (2) are more volatile and have a shorter onset of cooling sensation when administered by mouth or by inhalation. However, perceived potency and duration of the cooling sensory effect imparted by compounds of formulae (1) and (2) varies among genetically different individuals and therefore may be subject to analytical error or subjective assessment, as well as subject to influence due to regular use of unrelated cooling sensate compounds and formulations.

It has also been found that cooling sensory properties are attributable to derivatives of compounds of formulae (1) and (2), such as enol ether compounds of formulae (3) and (4):

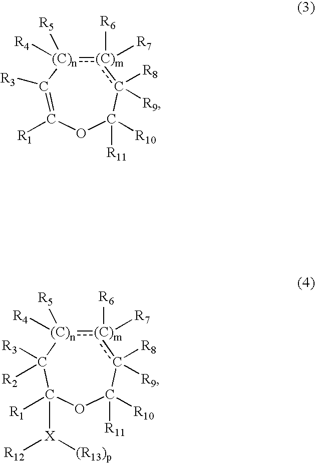

wherein R₁ though R₁₁, m, n, and the dashed bonds are defined as above for compounds of formulae (1) and (2) above; X is an oxygen, sulfur or nitrogen atom; and p is 0 when X is oxygen or sulfur, and 1 when X is nitrogen. R₁₂ and R₁₃ are each independently selected from linear, branched, or cyclic alkyl, alkenyl, aryl, aralkyl, acyl, or oxoacyl groups, a fragment of a dihydric or polyhydric alcohol, a carbohydrate fragment, a fragment of a di- or polycarboxylic acid, a fragment of an aminoacid, a fragment of a polypeptide, a polyether fragment, a polyester fragment, or a hydrogen atom.

The compounds of formulae (3) and (4) can be used in various formulations as precursor compounds capable of forming compounds of formulae (1) and (2) upon exposure to various factors such as moisture, heat, light, solvents, acids or bases, or enzymes. Such properties are desired in formulations wherein a delayed or stimuli-induced release of the cooling sensate compound is sought.

The carbonyl groups of compounds of formula (1) can also be modified to readily hydrolysable groups such as Schiff bases and enamines by reaction with various amines, and to oxazolidines and oxazolidinones by reaction with aminoalcohols and aminoacids. Such derivatives are also suitable precursors of compounds of formulae (1) and (2) for the purposes of imparting a cooling sensory effect, and use of such derivatives is fully within the scope of the present invention.

In addition to the desired cooling sensory effect, many of the compounds of formulae (1) though (4) typically possess attractive menthol-like scents of moderate or high potency, and the scent character of such compounds, depending on particular combinations of substituents R₁-R₁₁ and stereochemical composition, varies and includes unique combinations of woody, camphoraceous, fruity, tobacco-like, berry-like, animalistic, musty, floral or herbal notes. Such scent properties are useful to impart various characteristics to flavor or fragrance formulations and compositions, depending on the nature of the specific applications where such fragrances or flavors are used. However, the sensory thresholds of the perceived scent and taste characteristics attributable to compounds (1) through (4) are typically significantly above the thresholds for the cooling sensory effects. Thus, the perception of the scent and taste characteristics of compounds (1) through (4) will depend greatly on the amounts of compounds administered.

Compounds of formulae (1), (2), (3) and (4), depending on their specific chemical structure, physical conditions, the separation methods used to prepare them, the base composition used in formulating such compounds, and the like may exist in a substantially pure state, or as a mixture of interconvertible isomers, equilibrated or non-equilibrated. In addition, in the compound of formula (4), when R₁₂ or R₁₃ is a polypeptide or fragment thereof, the polypeptide or fragment thereof can represent one ore more cold receptor proteins present in a contacting tissue of a human or animal subject. Such a variation of compound (4) can form reversibly (or irreversibly) upon contacting any of the compounds of formulae (1), (2), (3), (4) with such receptors to cause a potent and long lasting cooling sensory effect.

Compounds of formulae (1) through (4) are also capable of causing secondary sensory effects when administered in quantities either above or below certain sensory threshold levels, and such secondary sensory, physiological or behavioral effects can stimulate feeling of freshness, a desire to breath deeply, excitement, cough suppression, irritation suppression, pain suppression, and the like, and thus are also useful in formulated products where such secondary effects are sought or welcomed by the consumer.

The compounds of formulae (1) and (2) can be synthesized from a variety of known starting materials using combination of reactions that typically employ conditions and reagents known in the art. Compound (2) can be prepared from compound (1) typically by using an acid catalyst or a metal alkoxide catalyst in a variety of solvents, including water or organic aprotic solvents, as compounds (1) and (2) are typically in an equilibrium in such solvents.

The compounds of formula (3) can be prepared by treatment of compounds (1) or (2), or mixtures thereof, under conditions providing for dehydration of the semi-ketal to the enol ether, typically in the presence of an acid catalyst, and optionally by heating. The compounds of formula (4) can be prepared from any of the compounds (1), (2), or (3) by reacting these compounds, typically in the presence of an acid catalyst and a suitable solvent, with a large variety of hydroxyl, carboxyl, thiol, or amino compounds. Certain of the compounds (1), (2), (3) and (4), have been found to be sufficiently stable and thus amenable to separation by methods known in the art, including chromatography. It has been found that in such cases, where separation of these compounds is possible, the cyclic semi-ketal or semi-acetals of formula (2) had the most potent cooling sensory effect with a shorter onset of action, as compared to the corresponding open form of formula (1) and to the ether form of formula (3).

The specific methods of synthesis for compounds (1) and (2) depend on the particular combinations of substituents R₁ though R₁₁ that are present in the structures of these compounds. Such methods can vary and one of ordinary skill in the art can introduce many variations in such methods in order to prepare a large variety of different compounds of formulae (1) and (2).

Disclosure of specific examples of synthetic methods will now be provided. These examples should not be construed as limiting.

In one embodiment, cooilng sensate compounds of formulae (7) and (8) are prepared by direct addition of an excess of methylmagnesum halide to either of the enantiomers of ketocampholenic acid (5):

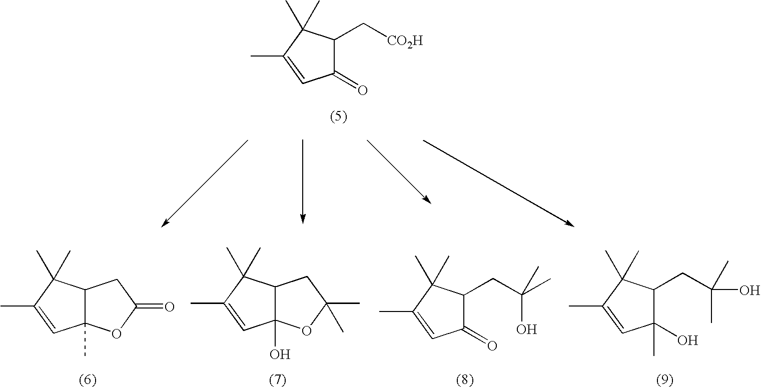

Such reaction typically yields a mixture of compounds, with a lactone of formula (6) being the predominant product upon isolation of the products from the reaction solution. The precise ratio of the products depends on the solvent used and the method of isolation. Typically, the reaction is carried out in a tetrahydrofuran or methyl tert-butyl ether (MTBE) solution of compound (5) at temperatures below 40° C., preferably at temperatures between 0 and 5° C. Typically, 2 to 6 equivalents of methyl magnesium chloride solution in tetrahydrofuran are added over a short period of time, and the resulting reaction mixture is quenched with a sufficient amount of alcohol, and then water, and the tetrahydrofuran is distilled out under reduced pressure. The residue is then diluted with water, carefully neturalized by adding dropwise dilute hydrochloric acid with continuous stirring to a pH of about 7-9, and then extracted with hexane or MTBE. The extracts, upon removal of the solvent, typically contain 50 to 98% of lactone (6) and various amounts of compounds (7), (8) and (9), each accounting for 0.5 to 15% of the reaction product mixture by weight. The resulting oily product mixture has a menthol-like, woody, mahogany-like, tobacco like, somewhat fruity odor, with a very powerful cooling effect instantly detectable in the nasal cavity upon olfactory evaluation. Upon separation of the compounds by chromatography, it has been found that pure compound (6) possesses a very agreeable and pleasant woody, mahogany-like, tobacco like, somewhat fruity odor, but lacks the cooling sensory effect. Compound (9) is practically odorless and tasteless. The powerful cooling effect us associated mostly with pure racemic compound (7), while racemic compound (8) has only a relatively weak cooling effect.

Upon treatment with dilute aqueous hydrochloric acid, both pure compounds (7) and (8) are rapidly and completely converted to a practically pure ketone of formula (10): 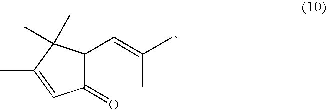
which is completely devoid of a cooling sensory effect and has very agreeable berry-like, raspberry-like odor and taste. Upon treatment of compound (9) with refluxing 0.1% aqeous sulfuric acid, an odoriferous furan of formula (11) is formed:

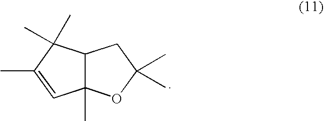

The compound (11) has a characteristic woody-ambery agreeable odor and is devoid of cooling sensory properties.

In a second embodiment, the compounds of formulae (7) and (8) are prepared by addition of methylmagnesium halide to derivatives of ketocampholenic acid (5), wherein the carbonyl group of the cyclopentenone ring is protected as a secondary enamine, and the carboxyl group is converted to an ester (typically an ethyl ester), compound of formula (12):

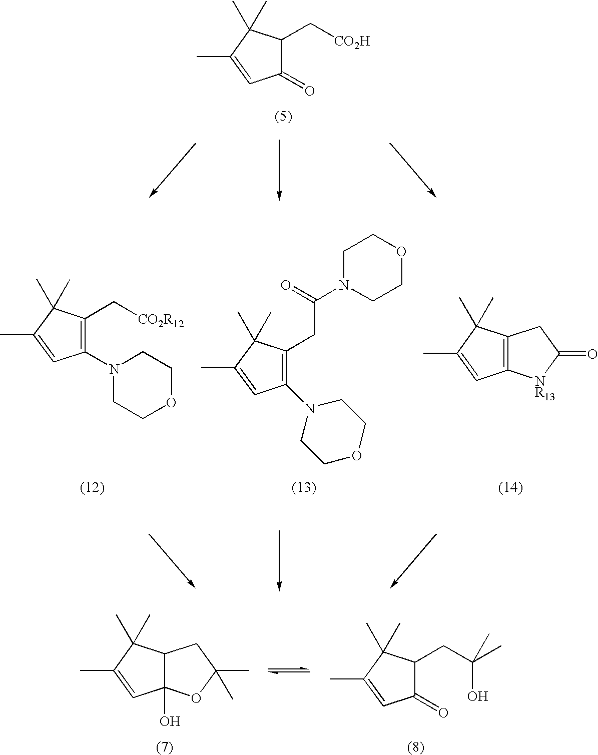

The compound of formula (12), wherein R₁₂ is typically an alkyl or cycloalkyl group having from 1 to 10 carbon atoms, is typically prepared by refluxing an ester of acid (5) with excess morpholine (other secondary amines, typically pyrrolidine, or piperidine, or dialkylamine, or diarylamine can also be used for this reaction), in the presence of a suitable solvent (typically, toluene or xylene), catalytic amounts of acid (typically, p-toluenesulfonic acid), preferably, in the absence of oxygen (typically, under nitrogen or argon), and under conditions allowing for removal of water forming in the reaction, followed by removal of the morpholine and the solvent under reduced pressure.

Alternatively, the carboxyl group is converted to a secondary amide of formula (13) under conditions substantially similar to those described above for making compound (12), except the acid (5) is used instead of an ester of acid (5). Alternatively, treatment of acid (5) with a primary amine (preferably, a linear, branched, or cyclic amine having from 4 to 15 carbon atoms, e.g., 1-octylamine) results in the formation of a bicylic enamine-amide of formula (14).

Addition of excess (typically 2 or more equivalents) of methylmagnesium halide to any of the compounds (12), (13), or (14) results in the formation of product mixtures, which upon hydrolysis contain typically 15-40% of racemic compound (7) and 5-20% of racemic compound (8), which can be readily purified by chromatography. The purified compound (7) prepared by such methods has a very powerful cooling sensory effect with a very weak menthol-like scent, while purified compound (8) is practically odorless and has a weak cooling effect.

In a similar embodiment, when either enantomer of 3-oxo4,5,5-trimethyloxopentaneacetic acid is protected as an enamine-ester, or enamine-amide, addition of methylmagnesium halide results in the formation of racemic compounds of formulae (15) and (16):

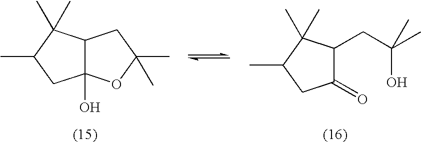

The purified compound (15) has a moderate to strong cooling sensory effect and a potent menthol-like, woody odor, while purified compound (16) is practically odorless and has a weak cooling sensory effect.

In a third embodiment, novel cooling sensate compounds of formulae (18), (19), (21), (22), (23), and (24) are prepared according to the following reaction scheme:

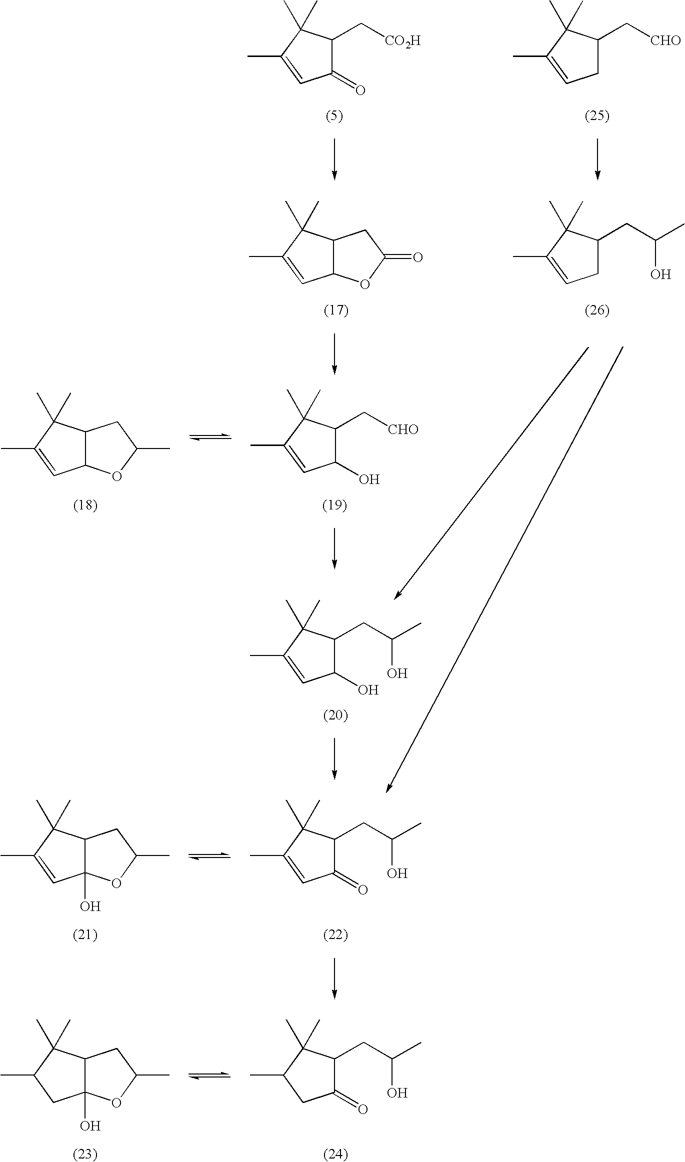

In this embodiment, acid (5) is converted to a known lactone (17), typically, by reduction of its sodium salt with sodium borohydride in methanol and in the presence of effective amounts of CeCl₃ or other cerium salt. The lactone (17) is then reduced to a mixture of lactol (18) and hydroxyaldehyde (19), typically by using a dialkyloxylithium hydride, or, preferably, a trilakyloxylithium hydride, such as di- or tri-ethoxy-, or di- or tri-buthoxy-lithium hydrides. The compounds (18) and (19) are then further converted to a diol of formula (20), typically, by addition of one or more equivalents of methylmagnesium halide. The diol (20) is then oxidized to a mixture of semi-ketal (21) and hydroxyketone (22). The selective oxidation is typically accomplished by using manganese dioxide as an oxidant in a suitable solvent such as methylene chloride. Catalytic hydrogenation, typically using a palladium, ruthenium, or platinum catalyst, allows for preparation of the saturated compounds of formulae (23) and (24).

Alternatively, campholenic aldehyde (formula 25), readily available on an industrial scale by rearrangement of alpha-pinene epoxide, is subjected to the addition of methylmagnesium halide to produce methylcampholenol of formula (26) as a mixture of isomers. The compound (26) is then oxidized to diol (20) by using, for example, selenium dioxide as an oxidant or as a catalyst in the presence of peroxide, or, alternatively, oxidized directly to a mixture of products containing desired compounds (22) and (23), typically by using air or oxygen, and typically under conditions favoring formation of singlet oxygen species, such as photosensitization and other methods known in the art.

The synthetic methods disclosed in this embodiment allow for preparation of either enantiomer of compounds (18), (19), (21), (22), (23), and (24), and, if so desired, any of these compounds can be racemized in the presence of a base or an acid in an aqueous solution.

In a fourth embodiment, preparation of novel cooling sensate compounds of formulae (30), (31), (32), and (33) is carried out according to the following reaction scheme: 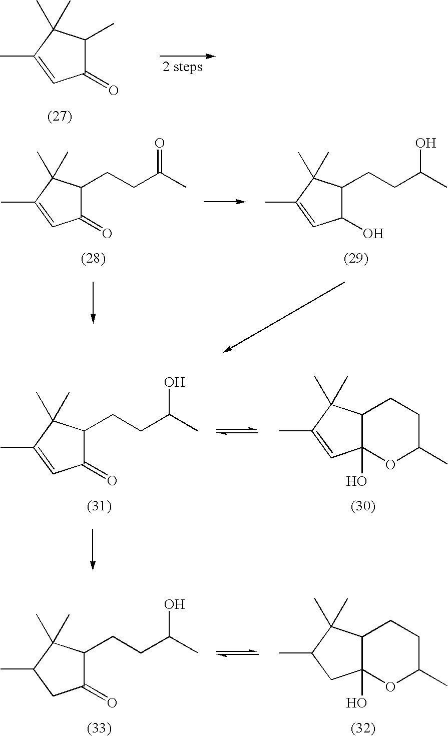
In this embodiment, diketone (28), which is readily available from a cyclopentenone of formula (27), is reduced to diol (29), which is then selectively oxidized, typically, by using manganese dioxide as an oxidant to a mixture of desired compounds (30) and (31). Alternatively, diketone (28) is directly reduced at the less hindered position to the mixture of compounds (30) and (31). Such reduction can be carried out to produce, preferentially, desired stereoisomers, and can be carried out enzymatically or microbially by methods known in the art. The double bonds in compounds (30) and (31) can be reduced to yield compounds (32) and (33), respectively. The reduction of the double bonds is typically carried by catalytic hydrogenation by using one or more stereoselective or non-stereoselective catalysts well-known in the art.

In a fifth embodiment, various cyclopentanones, such as dimethyl- or trimethyl-cyclopentanones, herein exemplified by 3,3,4-trimecylcyclopentanone (34) (readily available by hydrogenation of 3,4,4-cyclopent-2-enone), are converted via an enamine, exemplified by a morpholino-emanime compound (35), to compounds of formulae (36) and (37) according to the following reaction scheme:

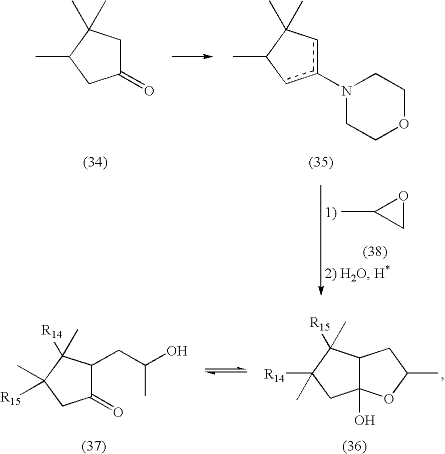

wherein R₁₄ is H and R₁₅ is CH₃, or R₁₄ is CH₃ and R₁₅ is H, and wherein any one of the dashed bonds is double and another is single.

In this embodiment, treatment of cyclopentenone 34 with a secondary amine, typically, morpholine, pyrollidine, piperidine, dialkylamine, diaryl amine, in the presence of an acid catalyst and under conditions favoring removal of water formed in the reaction, allows for preparation of a mixture of isomeric enamines of formula (35)., Such enamines can be alkylated with an epoxide, preferably, with either enantiomer of propylene oxide (38) in a known reaction readily taking place under elevated pressure and elevated temperatures, typically in the 100 to 200° C. range, and typically carried out in a sealed pressurized vessel. Upon cooling the reaction mixture and aqueous work-up in the presence of acid, followed by removal of the amine, a mixture of desired compounds of formulae (36) and (37) is produced.

This synthetic method is also readily applicable to the synthesis of novel cooling sensate compounds of formulae (41) and (42) from readily available dihydroisophorone (39) according to the following reaction scheme: 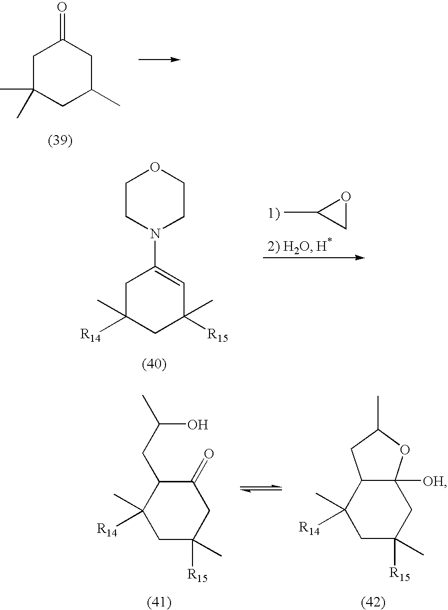
wherein R₁₄ and R₁₅ are defined above and conditions of the synthesis are substantially similar to those described for the preparation of compounds (36) and (37).

In a sixth embodiment, cooling sensate compounds (47), (48), (49), and (50) are prepared from a readily available and inexpensive isophorone (43) according to the following reaction scheme: 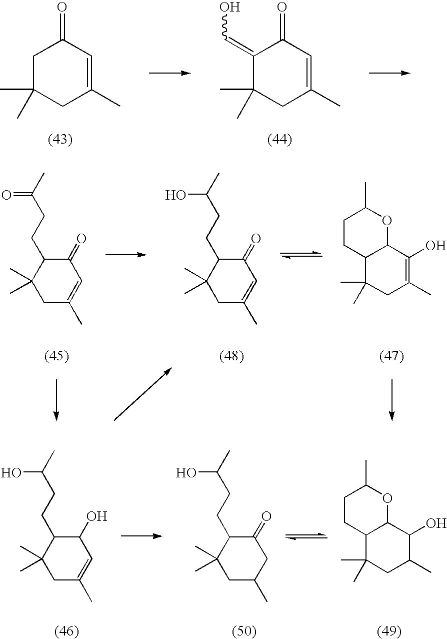
In this embodiment, isophorone (43) is converted to the diketone of formula (45) by alkylation of intermediate enolone (44), typically by using methylvinyl ketone. The intermediate enolone (44) is typically prepared by condensation of isophorone with formate esters, typically, with ethyl formate, in the presence of sufficient amount of strong base under conditions substantially similar to those known in the art for preparation of diketone (28) from cyclopentenone (27). The diketone intermediate (45) is then converted to compounds (47), (48), (49), and (50) using methods described above for the synthesis of compounds (30) through (33) from the diketone (28).

In a seventh embodiment, potent cooling properties of compounds (51), (52) and (53), which are compounds known in the art, are described: 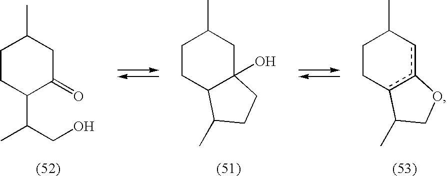
wherein any of the two dashed bonds ins double, and the other is single.

These compounds exist as a complex self-equilibrating mixture and such mixture, whether equilibrated nor not, has potent cooling properties when administered orally, upon inhalation, or when applied in the skin. Such compounds can be prepared by methods known in the art. More conveniently, these compounds are readily prepared by partial hydrogenation of natural menthofuran (54) using a platinum on carbon or a palladium on carbon catalyst according to the following scheme: 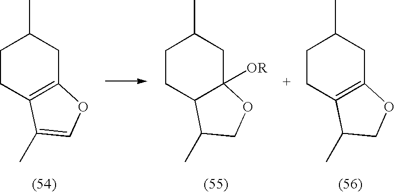
The partial hydrogenation reaction is typically carried out using an alcohol as solvent (typically, methanol), and, to avoid overhydrogenation, in the presence of catalytic amounts of acid, typically, acetic acid. The hydrogenation yields a complex mixture of stereoisomers of ketal (55) and enol ether (56), which upon removal of solvent are dissolved in acidified water under reflux for 30 minutes. After hydrolysis, the aqueous solution is adjusted to a pH of about 8-8 and extracted with MTBE or hexanes, to afford an organoleptically acceptable mixture of compounds (51) and (52), which upon standing equilibrates to a mixture that, in addition to the latter compounds, also contains isomers of enol ether (53).

In an eighth embodiment, cooling sensate compounds similar in potency and organoleptic properties to the above compounds (51), (52), and (53) are prepared from readily available pulegone (57) via alkylation of an isopropylidene sidechain using vinyl or allyl organometallic reagents (typically organo-copper reagents) and conditions known in the art. The synthesis is carried out according to the following scheme: 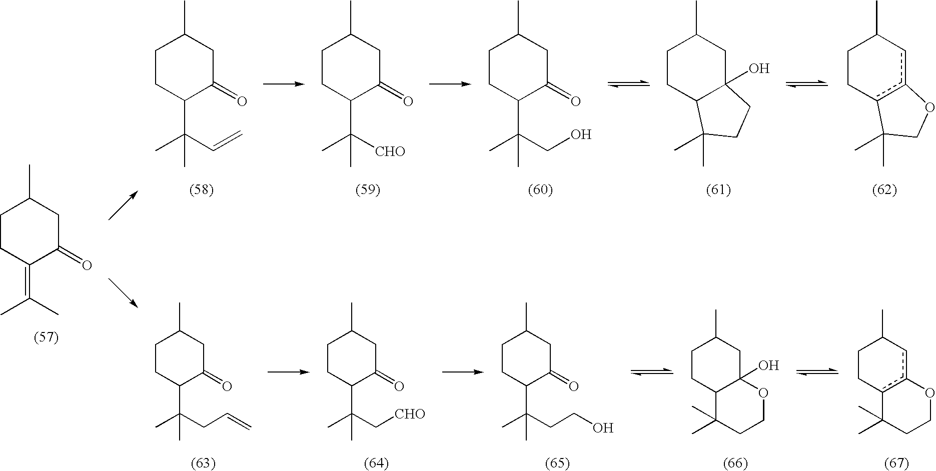
In this embodiment, the double bond of the enones (58) and (63) is typically cleaved by ozonolysis in methanol, followed by a reductive work-up with aqueous alkaline sodium thoisulfate. The aldehyde group in the resulting ketoaldehydes (59) and (64) is readily amenable to selective reduction, thereby affording corresponding sets of the mixtures of the desired compounds (60) through (62) and (65) through (67).

In a ninth embodiment, cooling sensate compounds are prepared by alkylation of readily available menthone, pulegone, carvone, carvomenthone, dihydrcarvone, or carvoneacetone. In this embodiment, the cyclohexane ring is alkylated at the alpha position with respect to the carbonyl group with a side chain, allowing for construction of a hydroxyketone compound with the hydroxyl group capable of forming a semiketal with the carbonyl group present in the starting materials. The alkylation can be carried out by a variety of methods known in the art. For example, alkylation can be carried out using suitable enamine compounds derived from the starting ketones, and alkylating reagents such as epoxides, aldehydes and ketones, acrylonitrile, acrylate esters, and halogenated compounds. This embodiment allows for synthesis of a large set of cooling sensate compounds of formulae (1) trough (4). Non-limiting examples of cooling sensate compounds resulting from alkylation of menthone and pulegone are shown below: 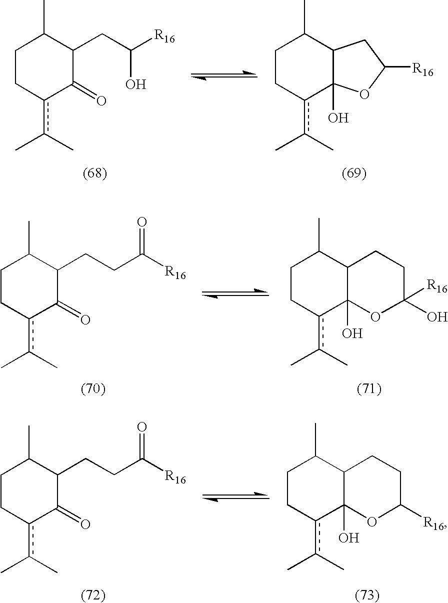
wherein R₁₆ denotes H or a linear or branched alkyl having from 1 to 6 carbon atoms.

The above described embodiments disclose cooling sensate compounds wherein the corresponding cyclic semi-ketal or semi-acetal form of formula (2) has at least one other cyclic fragment in the structure (i.e. wherein any of the two groups selected from R₁ through R₁₁ are optionally connected to each other by one or more carbon atoms other than those shown in the formulae (1) through (4), or by one heteroatom selected from oxygen, nitrogen or sulfur atoms).

In a tenth embodiment, it has been found that certain compounds of simpler structures also possess valuable cooling sensory properties of variable potency and duration of action.

The non-limiting representative examples of such compounds include those showing in the chart below:

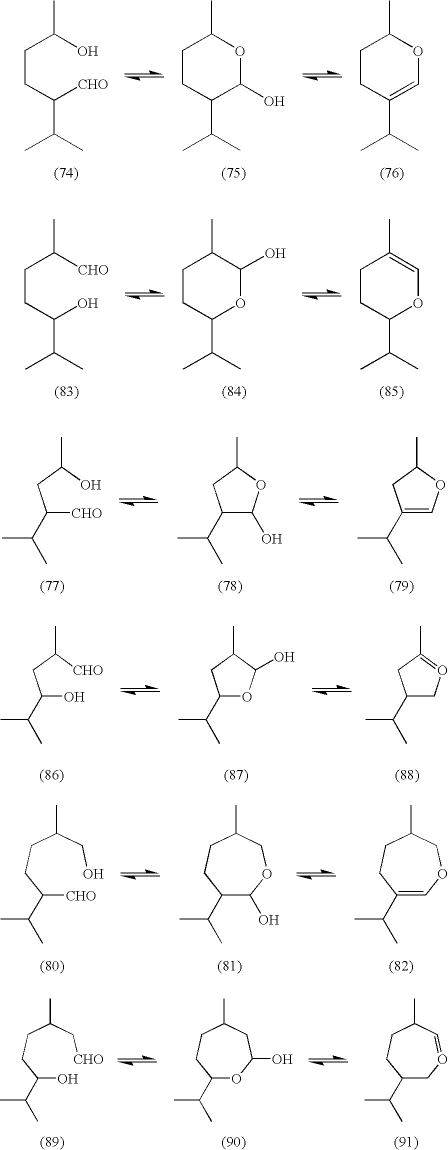

These compounds can be synthesized and used as pure enantiomers, pure stereoisomers, or mixtures of thereof. The methods of synthesis vary, depending on the compound desired. For example, compounds (74) and (75) can be prepared according the following reaction scheme:

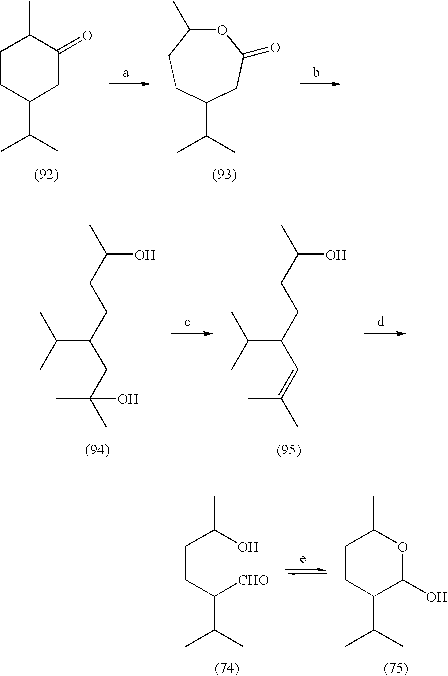

According to this reaction scheme:

(a) carvomenthone (92) is typically oxidized to lactone (93) using conditions and reagents that are ordinarily used in the art for performing Baeyer-Villiger oxidations. Such conditions can typically include organic peracids or suitable enzymes or microorganisms capable of such reaction with cyclic or linear ketones;

(b) the lactone (93) is then converted in a Grignard reaction to the diol of formula (94) using a sufficient amount of a suitable organometallic compound. Suitable organometallic compounds comprise compounds such as arylmagnesium halide, alkylarylmagnesium or methylmagnesium halide, or tertiary alkyl magnesium halide. Typically, 2 equivalents of organometallic compounds are required, and in practice, it is preferred to use about 2.0-2.5 equivalents. The reaction can be carried out with smaller or larger amounts of the organometallic compounds; however, the yields are lower if insufficient amounts are used, and a large excess of organometallic compound is wasteful;

(c) the diol (94) is then dehydrated to alcohol (95), typically in the presence of an acid, and optionally, by heating;

(d) the alcohol (95) is then oxidized by ozonolysis, typically in the presence of a suitable solvent such as dichloromethane or an alcohol such as methanol or other lower alkyl alcohol, and the ozonolysis reaction mixture is treated with a suitable reducing reagent such as dimethyl sulfide or other sulfur compounds. Preferably, the ozonolysis is carried out in methanol or ethanol, and the reaction products are then reduced using sufficient quantities of an aqueous solution of sodium thiosulfate, sodium sulfite, or mixtures thereof, wherein the aqueous solution is buffered with sodium bicarbonate or sodium hydroxide to a pH in the range from about 8 to about 11. After removal of the volatile solvents, the desired reaction product can be extracted using water-immiscible organic solvents such as ethers, hydrocarbons, esters, or halogenated hydrocarbons, and the resulting desired hydroxyaldehyde (74), or mixture of compound (74) and its semiketal (75), is therefore obtained;

(e) the resulting product (74) and (75), or a mixture thereof, can be equilibrated in the presence of a suitable solvent and an acid catalyst. The open (74) and cyclic (75) forms can be separated if desired; however, in practice it is not necessary to separate these forms. Heating or treatment of compounds (74) and (75) with a catalytic amount of acid results in the formation of enol ether (76).

In a variation of the above-described synthesis of compounds 74 through 75, the synthesis is carried out according to the following scheme:

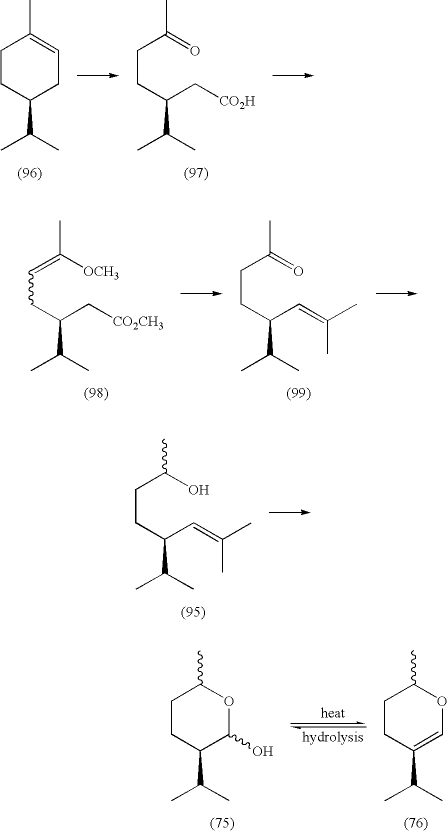

In this embodiment, 1-menthene (96) is oxidized to the keto-acid (97), typically by ozonolysis with oxidative work-up, or by ruthenium-tetroxide-sodium hypochlorite, or by alkaline potassium permanganate. The keto-acid (97) is then protected to form an enol-ether ester of formula (98) by refluxing in a mixture of methanol and trimethyl-orthoformate in the presence of catalytic amounts of tosic acid. 2.2 equivalents of methylmagnesium bromide are then added, and the resulting product is treated with acid to yield enone (99), which is then reduced by sodium borohydride in methanol in the presence of small amounts of CeCl₃. The entire sequence of reactions to the compound (95) can be carried out in high yield without purification of intermediates by chromatorgaphy or distillation. The compound (95) is then purified by chromatography and subjected to ozonolysis in methanol with reductive work-up using aqueous alkaline sodium thoisulfate, the methanol is removed under reduced pressure, and the desired compound (75) is easily recovered by extraction with MTBE in practically pure form (as mixture of stereoisomers). Heating of compound (75) in the neat state leads to predominant formation of enol ether (76), which is readily hydrolyzed back to compound (75) in the presence of water and traces of acid.

Compounds (74) and (75) can also be prepared from readily available isomers of piperitol (100) according to the following scheme:

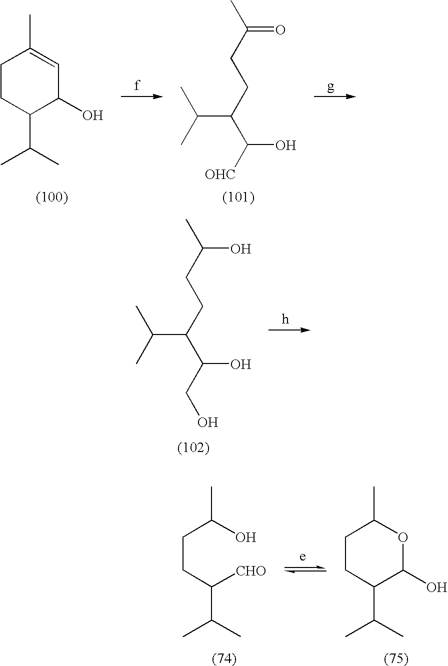

In this embodiment, several reactions are carried as follows:

(f) piperitol (100) is oxidized by ozonolysis with reductive work up to furnish hydroxylated ketoaldehyde (101) or a semiketal isomer thereof. Ozonolysis is preferably performed using methanol as a solvent, with reductive work-up using aqueous alkaline sodium thiosulfate;

(g) the resulting compound (101) is then reduced to the triol (102). Such reduction is typically carried out using sodium borohydride or by catalytic hydrogenation;

(h) the triol (102) is then cleaved with sodium periodate to produce compound (74), which equilibrates, depending on pH, to the oxamenthol (75).

In yet another embodiment, the oxamenthol (75) is prepared from the readily available known ketoaldehyde (103), which is an addition product of methylvinylketone to isobutyladldehyde or an enamine thereof. Such a synthesis is shown in the following scheme:

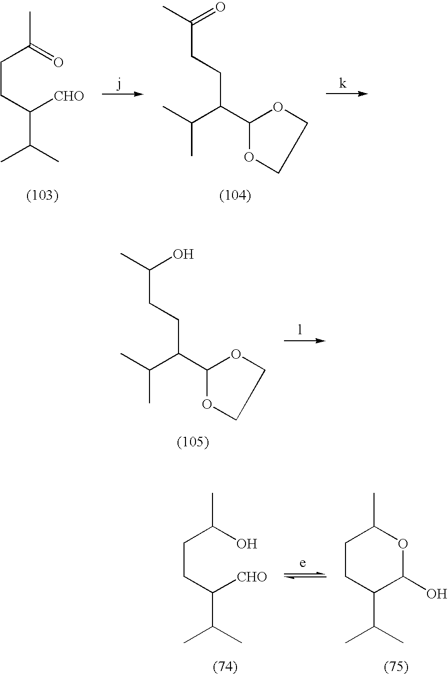

In this embodiment, either an enantiomer of ketoaldehyde (103) or a mixture thereof can be used. The synthesis is carried out as follows:

(j) ketoaldehyde (103) is selectively protected to form the keto-acetal exemplified herein by acetal (104);

(k) the carbonyl group of the keto-acetal (104) is then reduced to the hydroxy-acetal (105) by catalytic hydrogenation, or by sodium borohydride, or by other reducing reagents, optionally containing chiral catalysts. The keto group can also be reduced by baker's yeast or by other microorganisms, or by a dehydogenase enzyme, using methods and reagents known in the art. The resulting compound (105) is then readily deprotected under aqueous acidic conditions (stel 1) to furnish the desired compounds (74) and (75), along with enol ether (76).

Cooling sensate compounds (83), (84), and (85) are prepared from menthone (106) by using the reaction scheme shown below and reaction conditions substantially similar to those described above for the synthesis of compounds (74) and (75) from carvomenthone (92):

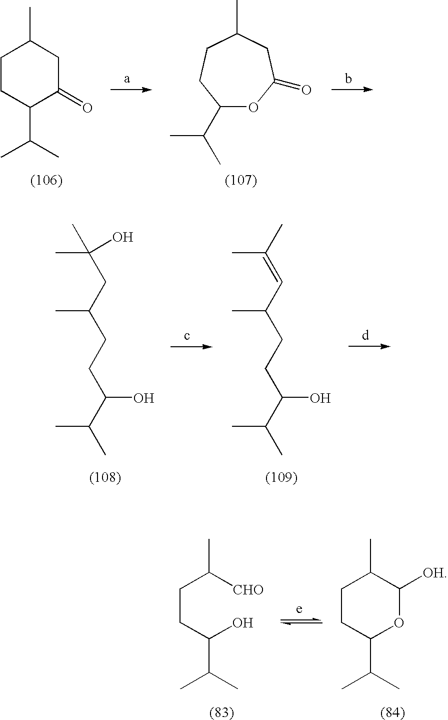

Compounds of formula (89), (90), and (91) are prepared using either enantiomer of 3-menthene via ozonolysis with reductive work-up to the corresponding ketoaldehyde compound, followed by protection of the aldehyde group, reduction of the carbonyl group, and deprotection according to the following scheme: 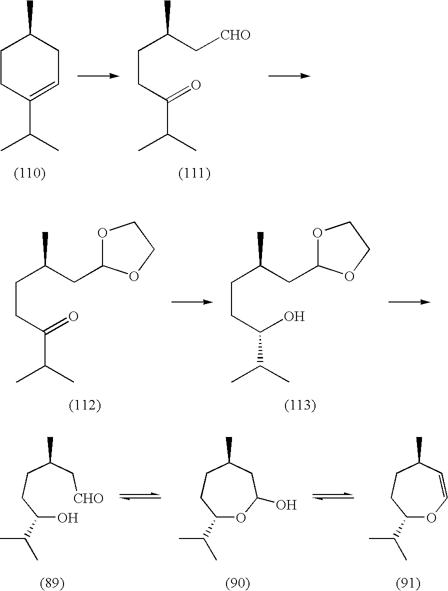
(preferred stereosiomers shown herein).

In a similar embodiment, the compounds of formulae (80), (81), and (82) are prepared from either enantiomer of 1-menthene in a reaction sequence substantially similar to that described above for compounds (89), (90), (91): 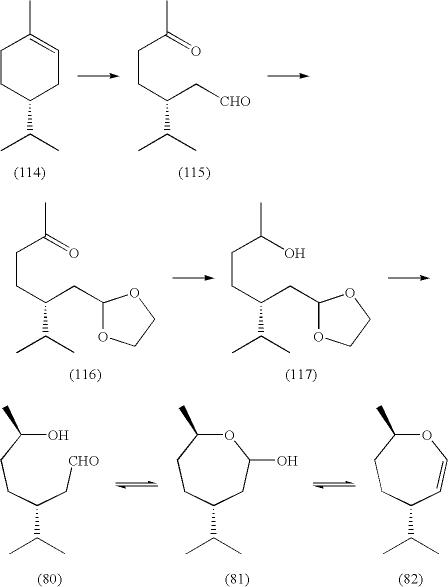
(preferred stereosiomers shown herein).

Alternatively lactols (81) and (90) are produced by reduction with lithium dialkoxy or trialkoxy hydrides of 7-methyl-4-isopropyl-ε-caprolactone and 4-methyl-7-isopropyl-ε-caprolactone, correspondingly.

Steroisomers of compounds of formulae (77) and (78) are produced from compounds (79) and (118) by hydrolysis.

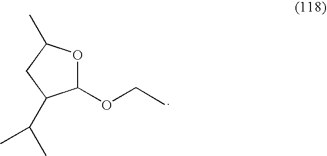

More conveniently, compounds (77), (78), and (79) are produced by reacting propylene oxide with the enamine reaction product of 3-methylburyraldehyde and a secondary amine, preferably, morpholine, pyrollidine, pyperidine, dialkylamine, diaryl amine, and the like. Such an alkylation reaction of enamines with epoxides is a known reaction and it typically is carried out at elevated pressures and temperatures.

The lactol compound (78) can also be prepared, for example, by reduction of lactone (119), 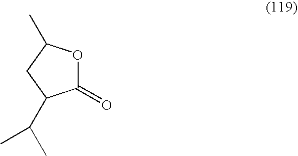
which, in turn, can be prepared from readily available gamma-valerolactone using reactions known in the art.

The compounds of formulae (1) through (4) are typically used in sensorily effective amounts in various formulations and base compositions where a cooling effect is desired to occur in the mouth, in the nasal cavity, in the lungs, or on skin or other epithelium. The compounds of formulae (1) through (4) can be used alone or in mixtures of other compounds known to cause a cooling sensory effect, such as menthol and other cooling sensate compounds.

The sensorily effective amounts of compounds (1) through (4) required to cause the desired cooling effect depend significantly on the particular nature of the compound, the substituents in the structure of these compounds, as well as on stereoisomer composition. Typically, depending on the application, and specific compound used, the amount of compounds (1) through (4) ranges from 1 nanogram to 100 milligram. The desired concentrations of compounds (1) trough (4) to be added to various base compositions can be readily established by one of ordinary skill in the art using known techniques comprising sensory evaluation of materials with various amounts of the cooling sensate compounds.

It has also been found that compounds of formulae (1) through (4) possess a synergistic effect resulting in a significant increase of intensity and duration of the cooling sensation imparted by various flavor formulations and products comprising menthol and/or other non-menthol cooling compounds. For example, the novel carbonyl compounds have been found to significantly increase the cooling mouth-freshening sensation of menthol-containing mouthwash, toothpaste, chewing gums, and mentholated or non-mentholated tobacco articles. The compounds are also useful in perfumery applications where a cooling refreshing effect is desired.

The compounds of formulae (1) through (4) have also been found to suppress irritation and cough caused by tobacco smoke when administered prior to smoking of tobacco, and therefore are useful as tobacco additives and in inhalation formulations for cough suppression. The cough suppression by compounds (1) though (4) was observed even when these compounds were administered in quantities below the apparent threshold level required to recognize a cooling effect caused by these compounds.

It is fully within the scope of the present invention to produce and use various proflavors and formulations and other derivatives that are capable of forming compounds of formulae (1) through (4). One of ordinary skill in the art can prepare and formulate such proflavor compositions and formulations that are capable of forming compounds of formulae (1) trough (4) upon exposure to moisture, light, heat, enzymatic activity, traces of acid or base, ultrasound, or by spontaneous decomposition or air oxidation.

In the foregoing embodiments, a number of novel campholenic and campholanic acid derivatives are disclosed. These compounds posess pleasant scents and are useful for flavor and fragrance applications. Also described herein are methods for preparing novel compounds by conducting carbon-carbon-forming reactions at various positions of 2-keto-4,5,5-trimethylcyclopent-3-eneacetic (formula 5), 2-keto-4,5,5-trimethylcyclopentaneacetic (formula 120), 3-keto4,5,5-trimethylcyclopentylacetic acids (formula 121), their salts, and derivative compounds modified at the carboxyl groups, such as various amides, N-hydroxyl compounds and the like:

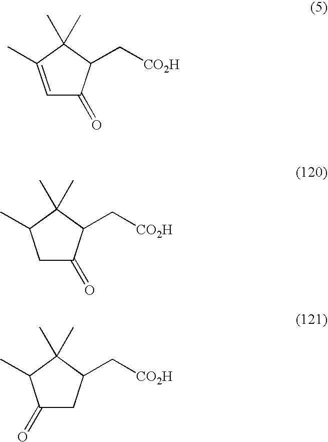

These compounds can be prepared by biological oxidation of camphor, bomeol, isobomeol, and their esters by known methods using one or more camphor-degrading microorganisms and their derivatives.

In an eleventh embodiment, novel derivatives of compounds (5), (120), and (121) are prepared using Grignard additions to the carbonyl groups of the cyclopentane ring. These additions proceed with high selectivity for the ketone function when the reactions are carried out using either suitable salts of the carboxylic compounds free acids or free acids. In the former case, these salts are formed in-situ when addition of the Grignard compound commences or when Barbier conditions are used.

Non-limiting examples of particularly useful salts are salts of alkali metals, alkali-earth methods, and quaternary amine compounds. However, other metal, amine, or ammonia salts, as well as various mixtures of salts of the above ketoacids, can also be used, wherein more than one cation-forming component is present, and/or more then one carboxylate or inorganic acid anion is present

The reaction scheme below provides summary of exemplary reactions and compounds formed from compounds of formulae (5), (120), and (121) by selective additions to the carbonyl group using Grignard or Barbier conditions: 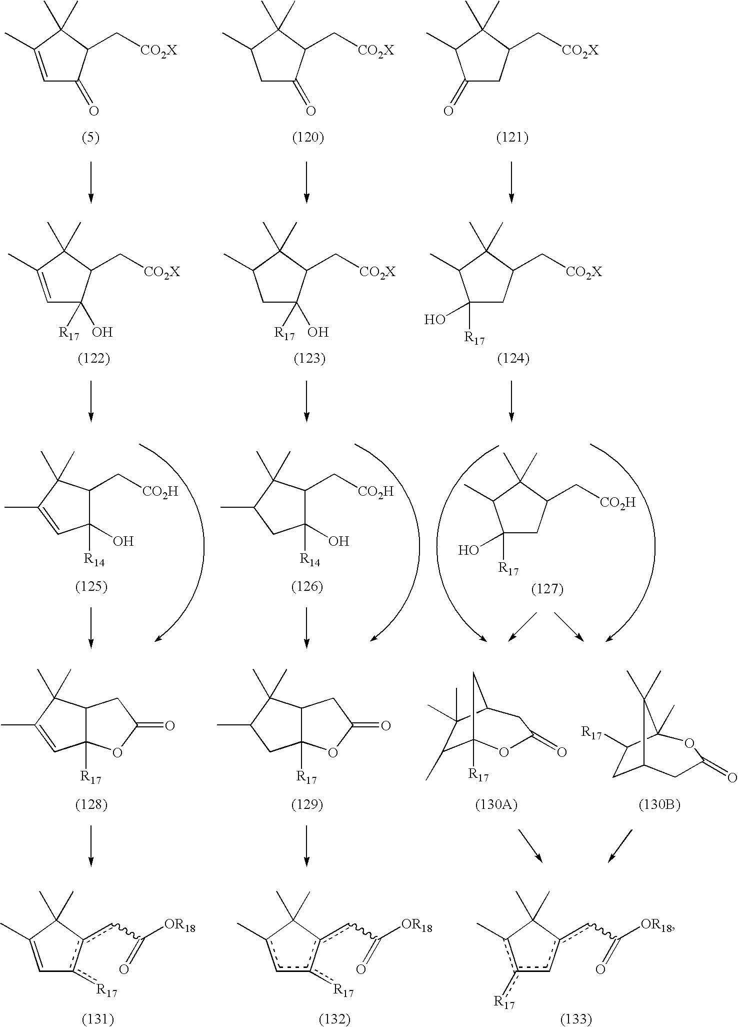
wherein:

X is H or a group selected from metal atom, NH₄, or fragment of quaternary amine, tertiary amine, secondary amine, primary amine, N-hydroxy compound, and

R₁₇ is a group having from 1 to 8 carbon atoms representing linear or branched or cyclic alkyl, alkenyl, alkynyl, and aralkyl groups, and

R₁₈ is selected from H or a group having from 1 to 8 carbon atoms representing linear or branched or cyclic alkyl, alkenyl, alkynyl, and aralkyl groups, and

one of the dashed bonds is double or single, and the others are single.

The salts of the ketoacids of formulae (5), (120), and (121) are novel compounds that typically can be prepared, for example, by reacting the free ketoacids with a suitable metal hydroxide or oxide compound, or with carbonate salts of alkali or alkali-earth metals, or with amino compounds. The salts of the ketoacids can also be prepared from carboxylic esters by saponification with a suitable base. The salts of keto acids can be prepared in a substantially anhydrous form, or as hydrates or solvates. Depending on the carbon-carbon bond forming reaction selected for addition to the carbonyl group, anhydrous salts or their hydrates or solvates can be used. For example, for conducting Grignard and Barbier reactions, substantially anhydrous salts are preferred to minimize consumption of reagents due to competing hydrolysis reactions. It has been found that substantially anhydrous alkali metal and alkali-earth metal salts of the above ketoacids are sufficiently soluble in dry aprotic solvents typically used for reactions with organometallic reagents, and Grignard and Barbier reactions can be carried out in a broad range of suitable solvents such as ethers, including tetrahydrofuran.

The salts of the ketoacids can be formed in a separate reaction, or they can be formed in situ in a suitable solvent, for example by adding a sufficient amount of a sacrificial organometallic compound reagent to a solution of free ketoacid in a suitable anhydrous solvent, such as tetrahydrofuran and other ethers, aromatic hydrocarbons, and like. When about one equivalent of a Grignard reagent is consumed, for example, an alkylmagnesium halide, the keto acids form a magnesium salt compound in-situ, which does not need to be isolated for subsequent addition reactions at the carbonyl group. The desired carbon-carbon bond forming reaction products can then be formed with a high selectivity and high yield by addition of about one additional equivalent of the same or different Grignard reagent. Such reaction products can be isolated as salts of formulae (122), (123), and (124), or as free acids of formulae (125), (126), and (127) or, upon acidification of the latter, as lactones (128), (129), (130A), and (130B). Among these compounds, cis-lactones of formula (128), wherein R₁₇ is selected from methyl or linear or branched alkyl or alkenyl having up to 4 carbon atoms, are of particular utility for use in fragrances and flavors, as such lactones possess very pleasant woody-fruity, mahogany-like, tobacco-like and somewhat coumarinic or floral odors. The formation of olefinic products of formulae (131), (132), and (133) may optionally be accompanied by a migration of the formed double bond, which is typically accomplished in the presence of an acid catalyst such as mineral acids, toluene sulfonic acid, and/or in the presence of an isomerization catalyst, such as a palladium salt, elemental palladium, palladium oxide, and the like. The extent of double bond migration and the composition of the olefinic product depends on (a) the particular substituents present in the compounds formed in the Grignard reactions, (b) the nature of the catalyst, and (c) the severity and duration of the treatment. The resulting olefinic compounds of formulae (131), (132), and (133) can also be hydrogenated to reduce one or more of the double bonds.

The esters (131), (132) and (133), wherein R₁₈ is a linear or branched alkyl or alkenyl group having from 1 to 5 carbon atoms, are of particular utility for use in fragrance and flavors. Their odors are characterized herein as very agreeable, pleasant floral, fruity, citrus-like, sweet, somewhat reminicent of known esters of alpha- and gamma-campholenic acids. These esters also induce a strong salivation upon tasting or smelling.

It has been found that lactones of formulae (128) and (129), as well as esters of formulae (131) and (132), and the corresponding free acids of the latter two compounds, can also be converted, selectively or non-selectively, to cyclic esters of formulae (134) and (135), correspondingly. 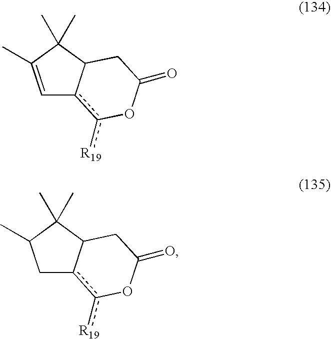
wherein R₁₉ is H or selected from a group having from 1 to 7 carbon atoms representing linear or branched or cyclic alkyl, alkenyl, or aralkyl groups, and wherein one dashed bond is single and the other is double or single.

These lactones posess pleasant and agreeable odors of moderate tenacity, reminiscent of those of the above-described lactones of formula (128).

The precise odor character depends on the particular structural variations of the compounds, such as the nature of R₁₉, the number and the positions of the double bonds, and the configuration of chiral centers.

In a twelfth embodiment, a group of novel odoriferous compounds is prepared from acids (5), (120), and (121) using synthetic methods including carbon-carbon-bond forming reactions at the substituent carrying the carboxyl group. In particular, compounds of formulae (136), (137), and (138) are provided: 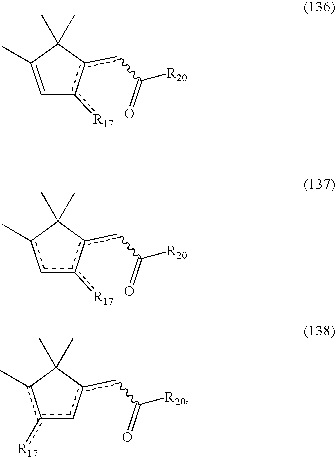
wherein R₁₇ and double bonds are defined above, and wherein R₂₀ is selected from H, or linear or branched alkyl or alkenyl groups having from 1 to 5 carbon atoms.

Particularly useful are variations of these compounds wherein R₁₇ is methyl and R₂₀ is methyl or 1-propenyl. These compounds have pleasant agreeable fruity-floral odors reminicent of irones, ionones, damascones, and damascenones.

Compounds (136), (137), and (138) are typically prepared by carbon-carbon bond forming reactions of cyclopentane compounds (128) through (133) at the side chain carrying the carboxyl-group. Such carbon-carbon bond forming reactions include, typically, Claisen condensations with excess of an alkyl ester of formic, acetic or an alkenoic carboxylic acid, in the presence of sufficient amount of base, typically an alkali metal alkoxide. The resulting products are then typically subjected to hydrolysis in the presence of acid, preferably strong inorganic acid, (exemplified herein by sulfuric acid) and optionally at elevated temperatures, until the desired decarboxylation of beta-ketoester adducts is substantially complete. The resulting compounds (136), (137), and (138) are then purified by distillation under reduced pressure.

Alternatively, compounds (136), (137) and (138), wherein R₂₀ is 1-propenyl or 2-methyl-1-propenyl, are prepared by adding about 2 equivalents of allyl or methallyl magnesium halide to the carboxyl group of compounds (128) through (133), and the resulting adducts are isolated and carefully heated in the presence of strong base such as potassium tert-buthoxide and a suitable aprotic solvent, such as dimethylformamide. After the loss of about one equivalent of propylene or isobutylene, the resulting adducts are acidified to isomerize the position of the double bond in the R₂₀ group, and to eliminate an additional equivalent of water if lactones (128), (129), and (130) were used as starting materials.

Alternatively, compounds (136), (137) and (138) are prepared by converting esters (131), (132), and (133), or free acrboxylic acids thereof, to secondary amides under conditions known in the art, for example, by heating with a secondary amine in the presence of a catalytic amount of acid and in a suitable solvent. Such amides are then subjected to addition of about one equivalent of a Gringard compound R₂₀MgHal, and upon hydrolysis in the presence of an acid, the mixtures of products containing compounds (136), (137), and (138) are obtained.

In a thirteenth embodiment, any of the compounds (5), (128), (129), (131), (132), and (133) are subjected to oxalation by using a sufficient amount of an oxalate ester (typically, diethyl oxalate) and a sufficient amount of a strong base (typically, sodium ethoxide). The oxalation proceeds with a very high degree of selectivity at the alpha position of the side chain carrying the carboxyl group in these starting materials. The resulting oxalation products are isolated and subjected to the addition of excess, typically 4 or more equivalents, methylmagnesium halide, and the resulting adducts are then treated with acid to cause partial dehydration of the product mixture.

The resulting product mixture has a woody-amber type odor with a degree of pleasant sweetness. When using compounds (5), (128), (129), (131), or (132) as starting materials for oxalation, followed by MeMgCl addition and by acidic dehydration, the final product mixture was found herein to contain several products comprising predominantly cyclic ethers of formulae (139), (140), and (141):

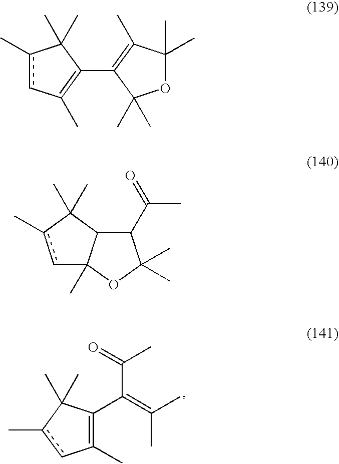

wherein the dashed bond is double or single.

When compound (130) or (133) was used, the resulting product mixture contained predominantly compounds of formulae (142) and (143):

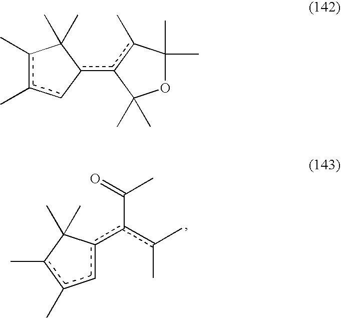

wherein any two of the dashed bonds are double and the others are single, but no two adjacent bonds are double at the same time.

### EXAMPLES

### Example 1

180 mg of ketocampholenic acid (5) (derived from biooxidation of R(+)camphor) were dissolved in 5 ml of dry tetrahydrofuran and stirred at room temperature (23° C.) under nitrogen. 1.3 ml of a 3M solution of methylmagnesium chloride was added dropwise over a period of 1 min. The temperature was allowed to rise to about 40° C. The reaction mixture was stirred for 10 minutes and quenched by dropwise addition of 1 ml of methanol. 5 ml of water was added, and the whole stirred and then extracted three times with 10 ml of hexane. The organic solvent fraction was dried over anhydrous sodium sulfate, filtered, and evaporated under reduced pressure to give 75 mg of a colorless oil. The oil had a menthol-like, woody, mahogany-like, tobacco like, somewhat fruity odor, and a very powerful cooling effect instantly detectable in the nasal cavity upon olfactory evaluation. The oil was analyzed by GC-MS and GC-FID and was found to contain approximately 85% of a cis-lactone of formula (6), along with small amounts of other compounds.

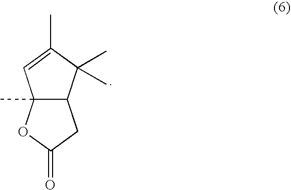

22 mg of the oil were dissolved in 10 ml of hexane, and 5 ml of 20% sodium hydroxide were added. The whole was intensely stirred for 30 minutes at room temperature. The hexane layer was collected and washed twice with 5 ml of saturated sodium bicarbonate, and then with 5 ml of water. The organic layer was dried over anhydrous sodium sulfate and evaporated under reduced pressure to give 7 mg of a clear colorless oil that slowly crystallized on standing. The oil was analyzed by GC-MS, GC-FID, and TLC, and was found to contain about 65% of compound (7) and about 30% of compound (8).

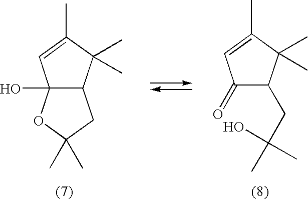

The resulting oil was found to have a characteristic menthol-like scent accompanied by a very powerful cooling effect in the nasal cavity when the odor of the neat sample was examined.

### Example 2

205 mg of 2-oxo-4,5,5-trimethylcyclopentylacetic acid (an 8:1 mixture of cis- and trans-isomers) were dissolved in 5 mL of dry tetrahydrofuran and stirred under nitrogen at room temperature (23° C.). 1.5 ml of a 3M solution of methylmagnesium chloride was added dropwise over a period of 2 min. The temperature was allowed to rise to about 35° C. The reaction mixture was stirred for 10 minutes and quenched by dropwise addition of 1 ml of ethanol. 5 ml of water was added, the stirred mixture was neutralized to a pH of about 8-9 by dropwise addition of a 5% solution of hydrochloric acid in water, and the whole stirred for 10 min. The resulting mixture was then extracted three times with 10 ml of hexane. The organic solvent fraction was dried over anhydrous sodium sulfate, filtered, and evaporated under reduced pressure to give 176 mg of a colorless oil with powerful characteristic menthol-like odor (fraction 2A). The crude oily product was analyzed by GC-MS and GC-FID, and was found to contain about 96% of lactone stereoisomers of formula (129): 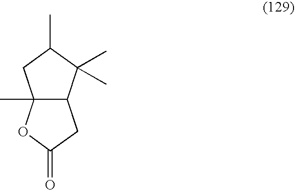
and about 3% of stereoisomers of compounds having formulae (15) and (16):

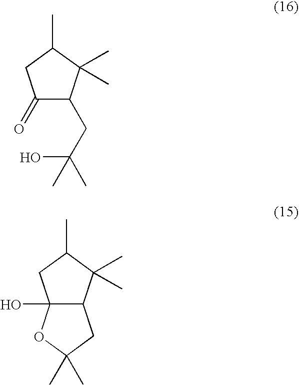

10 ml of 20% NaOH in water were added to the 1 ml of ethanolic solution of 120 mg of the oil resulting from the Grignard reaction, and the whole was stirred at room temperature for 1 hour. The reaction mixture was extracted two times with 10 ml of hexane, and the hexane layer was dried over anhydrous sodium sulfate, filtered, and evaporated under reduced pressure to give 4 mg of clear oil (fraction 2B) with a strong characteristic menthol-like, somewhat woody-camphoraceous odor, and with a powerful cooling sensory effect apparent upon smelling the sample.

The sample was analyzed by GC-MS, GC-FID, and TLC, and was found to contain a mixture of the stereoisomers of the formulae (15) and (16).

An analytically pure sample of a lactone having formula (129) was obtained by subjecting 30 mg of the fraction 2A sample to column chromatography on silica gel using hexane:ethyl acetate 8:1 as an eluent. The purified compound of formula (129) had a pleasant fruity-woody-coconut odor of moderate to weak strength and did not exhibit any significant ability to impart the cooling sensation akin to that observed upon olfactory evaluation of fraction 2B.

### Example 3

3.66 grams of compound (5), 10 ml of water, 5 ml of ethanol, and 1.70 g of sodium bicarbonate were stirred together at room temperature until evolution of carbon dioxide ceased (approximately 30 min). The whole was evaporated under reduced pressure at 85° C. until a constant weight was reached. The resulting transparent solid (4.4 g) was sodium ketocampholenate monohydrate. The compound was virtually odorless. When 0.1 ml of a 5% solution of sodium ketocampholenate was applied to the tongue, the compound was found to be virtually tasteless or slightly bitter, and caused a tingling-tangy sensation that lasted for about 10 min.

1.4 grams of sodium ketocampholenate monohydrate were dissolved in 10 ml of 70% ethanol and stirred at room temperature (25° C.). 1 gram of sodium borohydride (excess) was introduced over a period of 5 min and the whole was stirred. During the reaction the temperature of the solution was allowed to rise to about 40-45° C. After 1 hour of stirring, the mixture was cooled to 0-5° C., and the excess of borohydride was decomposed by dropwise addition of 5% aqueous hydrochloric acid until a pH of about 2-3 was reached and evolution of hydrogen gas ceased. The resulting solution was extracted three times with 20 ml of hexane containing 5% ethyl acetate. The organic layers were combined, dried over anhydrous sodium sulfate, and evaporated under reduced pressure to yield a white solid material. 20 ml of hexane were added to the solids, and the mixture was stirred for about 1 hour. The resulting solution was filtered and evaporated under reduced pressure to give 1.12 g of a 70:30 mixture of stereoisomers of lactones having formulae (17) and (143):

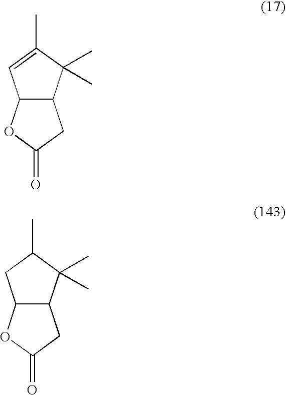

505 mg of the mixture of lactones were dissolved in 5 mL of dry tetrahydrofuran and stirred under nitrogen. 4 ml of a 3M solution of methylmagnesium chloride were introduced dropwise over period of 2 min and the temperature was allowed to rise briefly to about 40-45° C. The stirring was continued for 15 min at room temperature. The stirred reaction mixture was quenched by dropwise addition of 2 mL of absolute ethanol over 5 min, 10 ml of water was added, and the mixture was neutralized to a pH of about 8-9 by dropwise addition of 5% aqueous hydrochloric acid. The whole was extracted 3 times with 15 ml of methyl tert-butyl ether. The extracts were combined, dried over anhydrous sodium sulfate, and evaporated under reduced pressure to give 485 mg of a white crystalline solid which was found to be a 68:32 mixture of unsaturated and saturated diol stereoisomers of formulae (144) and (145):

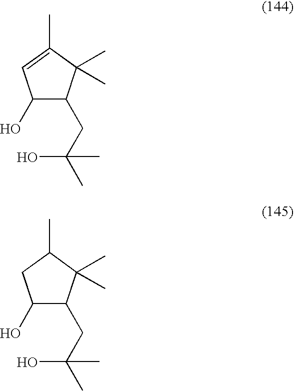

Approximately 180 mg of pyridinium chlorochromate in 10 ml of 1,2-dichloroethane were introduced dropwise to the solution of 150 mg of the mixture of compounds (144) and (145)in 10 ml of 1,2-dichloroethane and 0.2 ml of pyridine. The whole was stirred at 35° C. for 30 minutes, 0.5 ml of isopropanol were added, and the whole was stirred for another 30 min at room temperature. 200 mg of sodium bicarbonate were introduced and the resulting mixture was evaporated to dryness under reduced pressure. 10 ml of hexane and 2 ml of methyl tert-butyl ether were added to the dark solids and the whole was stirred for 20 min. The resulting solution was filtered and evaporated to dryness under reduced pressure to give 106 mg of a clear colorless oil with a powerful characteristic menthol-like somewhat woody odor and a very powerful cooling sensory effect that was instantly felt in the nasal cavity upon olfactory evaluation of the neat sample or sample transferred onto a smelling strip.

The resulting oil was analyzed by GC-MS and NMR, and was found to contain a 55:45 mixture of isomers of unsaturated and saturated hydroxyketones and semiketals of formulae 7, 8, 15, and 16.

In a variation of this example, a high purity lactone (17) was prepared by reducing the sodium salt of compound (5) in methanol with sodium borohydride in the presence of about 0.2 equivalents of CeCl₃. Addition of methylmagnesium chloride to the pure lactone (17) afforded practically pure diol (144), which was oxidized using manganese dioxide in methylene chloride to a practically pure hydroxyketone (8) with small amounts of semiketal (7).

### Example 4

2.46 g of ethyl 2-oxo-4,5,5-trimethylcyclopentylacetate (12:1 mixture of cis- and trans-isomers) and 20 ml of 20% sodium hydroxide were stirred together at room temperature for 1 hour, and then refluxed for 15 min. The resulting solution was washed 2 times with 20 ml of hexane, and hexane fractions were discarded. The aqueous solution was acidified by dropwise addition of 10% aqueous hydrochloric acid until a pH of about 2-3 was reached. The resulting mixture was extracted 3 times with 10 ml of ethyl acetate, and the extracts were combined, dried over anhydrous sodium sulfate, and evaporated under reduced pressure to give 2.05 g of 2-oxo-4,5,5-trimethylcyclopentylacetic acid (as a 4:1 mixture of cis- and trans-isomers), as a transparent oil that rapidly crystallized on standing.

1.02 g of 2-oxo-4,5,5-trimethylcyclopentylacetic acid, 10 ml of water, and 470 mg of sodium bicarbonate were stirred together for 20 minutes and then refluxed for 10 min. The whole was evaporated under reduced pressure and then dried in an oven at 105° C. to give 1.16 g of practically anhydrous sodium 2-oxo-4,5,5-trimethylcyclopentylacetate.

1.04 g of the resulting salt were dissolved in 10 ml of 70% aqueous ethanol and reduced by adding 1.02 g of sodium borohydride (excess was added). The reaction mixture was treated following the procedure for reduction of sodium ketocampholenate in Example 6 to give 0.84 g of the lactone of formula (143) as a mixture of stereoisomers.

### Example 5

0.5 mg of a mixture of compounds (7) and (8) prepared in Example 1 were dissolved in 1 ml of trimethyl orthoformate containing 0.05 mg of toluenesulfonic acid. The resulting solution was allowed to stand at room temperature for 30 minutes and then was analyzed by GC-MS and GC-FID. The analysis showed the presence of a single compound (over 98% purity by GC-FID) that was the ketone of formula (10).

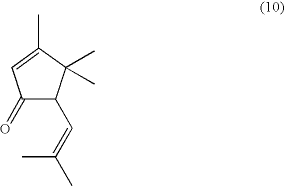

### Example 6

5 mg of a mixture of compounds (7) and (8) prepared according to Example 1 were stirred with 5 ml of water, followed by addition of 0.1 ml of 40% sulfuric acid. The stirring was continued for 30 min at room temperature. The resulting solution was neutralized by addition of excess sodium bicarbonate and the whole was extracted 2 times with 10 ml of hexane. The hexane extracts were combined, dried over anhydrous sodium sulfate, and evaporated under reduced pressure to give 3.6 mg of a clear colorless oil with a characteristic fruity-woody odor. The oil was analyzed by GC-MS and was found to contain a practically pure ketone of formula (10).

In comparison, when 2 mg of the mixture of compounds 111 and 121 were treated with 1 ml of 40% concentrated sulfuric acid by stirring for 15 min at room temperature, the product was a mixture of ketone isomers of formula (146). 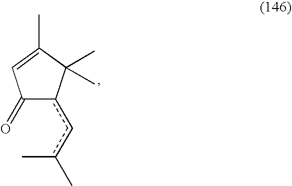
wherein any one of the dashed bonds is double and the other is single.

1 mg of a neat sample of compounds (7) and (8) were heated in a sealed glass vial at 250° C. by means of an oil bath for 5 minutes, and the vial was quickly cooled in an ice-water bath. The sample was analyzed by GC-MS,GC-FID, and GC-O, and was found to contain about 25% of ketone (10) and about 70% of a compound of formula (147), which had a characteristic woody-camphoraceous odor but no significant cooling sensory effect.

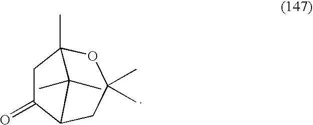

A 5 ppm solution of 98% pure ketone (10) in purified water was prepared. 10 ml of the solution was tasted by rinsing it in the mouth for 10 sec. The ketone (10) was found to have a pleasant fruity, berry-like taste reminiscent of raspberry, but had no discernible cooling sensory effect.

### Example 7

A series of solutions of a mixture of compounds (7) and (8) (approximately 60:40 ratio) were prepared in purified water for subsequent evaluation of cooling sensory effects and taste properties at different concentrations. The solutions were tasted by taking 10 ml of each sample in the mouth, rinsing the mouth for 30 seconds, and discarding the solution by spitting. The cooling sensory effects were recorded as extreme (5), strong (4), moderate (3), weak (2), or none (1), and the ranks were noted over a period of time of 4 hours. Only one sample was tasted per day. All samples were kept in a refrigerator until the taste evaluation and were brought to room temperature before tasting. The compounds (7) and (8) were found to impart powerful cooling properties over a broad range of concentrations tasted. No burning or painful sensations were experienced even when the cooling effect was ranked as extreme.

The results are summarized in Table 1.

TABLE 1

| Concen- | | | | | | | | | |
|---|---|---|---|---|---|---|---|---|---|
| tration | | | | | | | | | |
| of com- | | | | | | | | | |
| pounds | Time | | | | | | | | |
| (7) + (8), | 15 | 1 | 5 | 15 | 30 | 1 | 2 | 3 | 4 |
| Ppm | sec | min | min | min | min | hour | hours | hours | hours |
| 0 | 1 | 1 | 1 | 1 | 1 | 1 | 1 | 1 | 1 |
| 0.1 | 1 | 2 | 2 | 2 | 2 | 1 | 1 | 1 | 1 |
| 0.2 | 2 | 3 | 4 | 4 | 3 | 3 | 2 | 1 | 1 |
| 1 | 2 | 3 | 4 | 4 | 4 | 4 | 3 | 1 | 1 |
| 10 | 3 | 4 | 4 | 5 | 5 | 5 | 4 | 3 | 2 |
| 100* | 4 | 5 | 5 | 5 | 5 | 5 | 4 | 3 | 2 |

*a 5 ml sample of the solution at this concentration was tasted.

Freshly made solutions of compounds (7) and (8) were also found to be practically devoid of any other taste property or scent of menthol at the concentrations tested.

When aqueous solutions of these compounds in distilled water were stored for several weeks at room temperature and exposed to light, they retained the cooling sensory effect properties without a noticeable loss of potency. However, samples with initial concentrations of compounds (7) and (8) in excess of 10 ppm developed a pleasant raspberry-like taste in addition to the cooling effect Mass-spectral analysis showed gradual accumulation of small amounts of ketone (10).

### Example 8

A piece of spearmint-flavored chewing gum (Eclipse® brand, W.M. Wrigley Jr. Company, purchased in a local grocery store) was intensely chewed for 30 minutes, and then discarded. A strong cooling sensation was observed in the mouth during the first 15 minutes after the beginning of chewing. The sensation gradually became weak within the next 15 minutes of chewing. The weak cooling sensation lasted for an additional 10 minutes after discarding the gum.

2 hours after chewing the gum, the mouth was rinsed with 10 ml of the 10 ppm solution of a mixture of compounds (7) and (8) prepared according to Example 1. A strong cooling effect developed in the mouth. 2 minutes after rinsing the mouth, a fresh piece of the same brand of gum was intensely chewed for 30 minutes, and then discarded. During the entire chewing period, the cooling effect in the mouth was ranked as extreme. The extreme sensation continued for about 30 minutes after discarding the gum and then gradually diminished over the next 2 hours to non-detectable.

### Example 9

10 ml of “Blue mint” Target Corporation store brand antiseptic mouthrinse (purchased in a local Target store) with a menthol content determined to be 420 ppm was used to rinse the mouth for 30 seconds, and then discarded by spitting. The mouth was immediately rinsed twice with 25 ml of tap water. A moderate-to-weak cooling sensation was observed in the mouth for 10 minutes after rinsing.

8 hours after the first use of the mouthrinse, the mouth was rinsed with 10 ml of the 10 ppm solution of a mixture of compounds (7) and (8) prepared according to Example 1. A strong cooling effect developed in the mouth 2 minutes after rinsing the mouth with the solution of compounds (7) and (8). A fresh 10 ml sample of the above antiseptic mouthrinse was used to rinse the mouth for 30 seconds, after which the mouth was immediately rinsed twice with 25 ml of tap water. The cooling effect in the mouth was ranked as extreme for a period of 1 hour, and then gradually diminished over the next 2 hours to non-detectable.

24 hours after the experiment, a sample of the same brand antiseptic mouthrinse was supplemented with 3 ppm of a mixture of compounds (7) and (8). 10 ml of the sample were used to rinse the mouth as above. The cooling effect in the mouth was ranked as extreme for a period of 1 hour, and then gradually diminished over the next 3 hours to non-detectable.

### Example 10

2 g of toothpaste (Colgate® Total® Advanced Fresh gel, purchased in a local store), was used to brush the teeth in an ordinary way, and the mouth was thoroughly rinsed. The pleasant strong-to-moderate cooling sensation in the mouth lasted for approximately 15 min.

12 hours after brushing the teeth, the mouth was rinsed with 10 ml of the 10 ppm aqueous solution of a mixture of compounds (7) and (8) prepared according to Example 1. A strong cooling effect developed in the mouth. 2 minutes after rinsing the mouth, the teeth were brushed with 2 g of toothpaste as above, and the mouth was thoroughly rinsed. The extreme-to-strong cooling sensation lasted for approximately 2 hours and then gradually diminished over the next 1 hour.

### Examples 11-14

10 ml of 10 ppm of an aqueous solution of a mixture of compounds (7) and (8) prepared according to Example 1 were used to rinse the mouth. A strong to extreme cooling sensation was observed for a period of 1 hour. Next, 200 ml of unsweetened coffee (40-45° C.) was consumed within 15 min. The consumption of coffee did not diminish cooling effect in mouth.

In a separate experiment, small amounts of carbonated beverages were ingested periodically for 2 hours after oral application of compounds (7) and (8). The carbonated beverages were found to pleasantly rejuvenate and enhance the cooling and refreshing sensation effected by compounds (7) and (8).

In a separate experiment, the cooling sensation caused by rinsing the mouth with 10 ml of 1 ppm of compounds (7) and (8) was also found to be enhanced by smoking a non-mentholated cigarette.

In a separate experiment, smoking a mentholated cigarette after 2 hours of oral application of compounds (7) and (8) caused an extreme cooling effect in thee mouth that lasted for approximately 1 hour.

### Examples 15-16

After shaving the face under a warm and ample shower stream without soap, the right half of the face was moistened with 10 ml of a 10 ppm aqueous solution of a mixture of compounds (7) and (8) by means of a paper towel, while the left half was treated identically with purified water. A pleasant cooling effect was detected on the skin of the right half of face that lasted for about 15 minutes.

In a separate experiment, 0.2 mg of a mixture of compounds (7) and (8) in 0.5 ml of a USP-grade mineral oil were applied to the skin on the left side of the face in an area over the upper lip and on the cheek (over approximately 50 cm²), while identical areas of the face on the right side were treated with the USP-grade mineral oil without any supplements. A powerful cooling sensation was detected for approximately 30 minutes in the treated areas on the left side of the face, while no cooling effect was observed in the treated areas of the right side of the face. The diffusive powerful cooling effect was also detected in the left nostril.

Having described preferred embodiments of the present invention, it is to be understood that the invention is not limited to those precise embodiments, and that various changes and modifications may be effected therein by one skilled in the art without departing from the scope or spirit of the invention as defined in the following claims.

1. A composition comprising a base material and a compound having the formula: 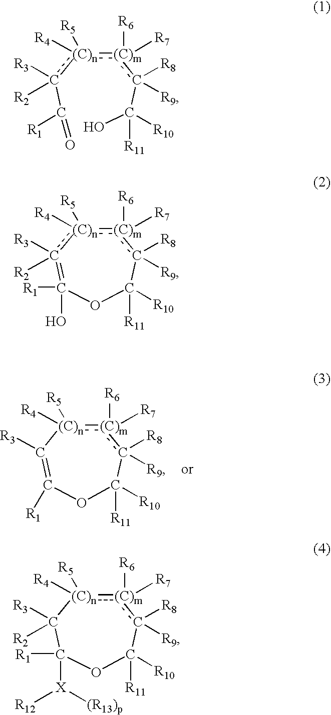
wherein R₁ through R₁₁ are each independently selected from H, linear or branched alkyl, alkenyl, or alkylidene groups having from 1 to 6 carbon atoms, or cycloalkyl or cycloalkenyl groups having from 3 to 10 carbon atoms,
R₁₂ and R₁₃ are each independently selected from linear, branched, or cyclic alkyl, alkenyl, aryl, aralkyl, acyl, or oxoacyl groups, a fragment of a dihydric or polyhydric alcohol, a carbohydrate fragment, a fragment of a di- or polycarboxylic acid, a fragment of an aminoacid, a fragment of a polypeptide, a polyether fragment, a polyester fragment, or a hydrogen atom;
n and m are each independently 0 or 1;
the total number of carbon atoms in the compound is less than 20; and
any one of the dashed bonds may be single or double, with the provisio that no more than one dashed bond is double, and no two adjacent bonds in the structures are double at the same time,
wherein the amount of the compound is selected such that the compound produces a cooling sensory effect when the composition is tasted, smelled, applied to the skin of a mammal, or applied to an internal epithelium of a mammal.

2. A composition according to claim 1 wherein at least one of the R₁ through R₁₁ groups further includes a group selected from hydroxyl, amino, carboxyl, and carboxamide groups.

3. A composition according to claim 1 wherein any two R groups may be connected to each other by one or more carbon atoms other than those shown in the formulae, or by a heteroatom selected from oxygen, nitrogen, and sulfur atoms.

4. A composition according to claim 1 wherein the compound comprises a compound having the formula 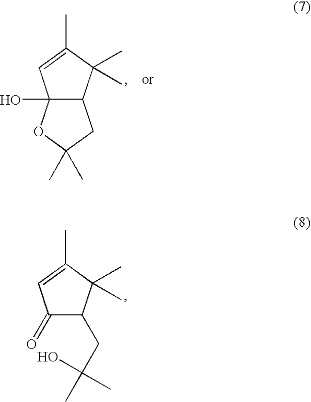
and combinations thereof.

5. A composition according to claim 1 wherein the compound comprises a compound having the formula 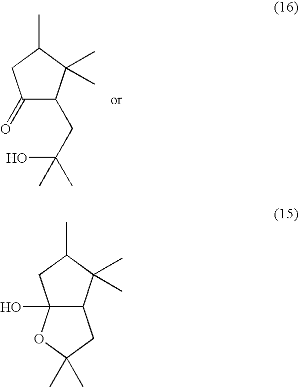
and combinations thereof.

6. A composition according to claim 1 wherein the amount of the compound is selected such that the compound also imparts a fragrance, flavor, or combination thereof to the composition.

7. A composition according to claim 1 wherein the composition is selected from the group consisting of chewing gums, confectioneries, and beverages.

8. A composition according to claim 1 wherein the composition is selected from the group consisting of toothpastes and mouthwashes.

9. A composition according to claim 1 wherein the composition is a smoking or chewing tobacco article.

10. A composition according to claim 1 wherein the composition is selected from the group consisting of cosmetics, skin care products, and skin cleaning products.

11. A composition according to claim 1 wherein the composition is a medicinal preparation.

12. A composition according to claim 1 wherein the compound produces a cooling sensory effect upon exposure of the composition to moisture, heat, light, solvents, acids, bases, or combinations thereof.

13. A method of producing a cooling sensory effect in a mammal comprising:
(a) providing a composition according to claims 1-12; and
(b) allowing the mammal to sample the composition by tasting it, smelling it, applying it to the skin of the mammal, applying it to an inner epithelium of the mammal, or a combination thereof.
